(19) United States
(12) Patent Application Publication
(10) Pub. No.: US 2005/0015096 A1
(43) Pub. Date: Jan. 20, 2005

(54) Tissue and membrane fixation apparatus and methods for use thereof

(76) Inventor: Dana A. Oliver, Jacksonville, FL (US)

Correspondence Address: EDWARDS & ANGELL, LLP, P.O. BOX 55874, BOSTON, MA, 02205, US

(21) Appl. No.: 10/623,170

(22) Filed: Jul. 18, 2003

(51) Int. Cl. A61B 17/58

(52) U.S. Cl. 606/104

(57) ABSTRACT

The present invention relates to surgical fastener systems, and more particularly to surgical fasteners in the form of tacks, and to apparatus and methods for highly reliable application of surgical fasteners for approximation and fixation of tissue and membranes in furtherance of surgical procedures (e.g., Autologous Chondrocyte Implantation) involving cartilage (e.g., knee cartilage).

### FIELD OF THE INVENTION

The present invention relates to surgical fastener systems, and more particularly to surgical fasteners in the form of tacks, and to apparatus and methods for highly reliable application of surgical fasteners for approximation and fixation of tissue and membranes in furtherance of surgical procedures (e.g., Autologous Chondrocyte Implantation) involving cartilage (e.g., knee cartilage).

### BACKGROUND OF THE INVENTION

The use of surgical fastening devices (e.g., sutures, staples, screws, clips, tacks and anchoring devices) in connection with surgical procedures is known. For example, commonly accepted protocol calls for the use of one or more of such fastening devices in connection with an Autologous Chondrocyte Implantation (ACI) treatment to repair knee cartilage.

According to such protocol (as outlined in, for example, Brittberg et al, “Treatment of Deep Cartilage Defects in the Knee With Autologous Chondrocyte Transplantation”, New England Journal of Medicine, 331:889-895 (Oct. 6, 1994) and Minas et al, “Chondrocyte Transplantation”, Operative Techniques in Orthopaedics, Vol. 7, No. 4, pp. 323-333 (October 1997)), a patient's knee is surgically entered to remove a biopsy of healthy cartilage tissue from a cartilage defect site. The healthy tissue is then cultured externally, causing the healthy cartilage cells to multiple. Later, the knee is once again entered, and the defect site is prepared for treatment by removing the damaged cartilage and measuring the lesion size. Treatment then entails placing the cultured cartilage into the lesion, which is then sealed off with a natural or synthetic membrane patch (e.g., a periosteum patch) using the one or more surgical fasteners. Within the lesion, the healthy cartilage cells multiply and integrate with surrounding cartilage such that over time, the healthy cells mature and fill in the lesion with healthy cartilage.

Initially, it was believed that sutures should be used to seal off the membrane patch, wherein the sutures would be used to sew the patch atop the cartilage. And although suturing the patch in place as such generally produced a desired result (i.e., reliable attachment of the patch to the cartilage), the suturing instruments were quite small, and thus difficult to grasp and maneuver with the level of precision required for the procedure. In turn, the suturing process was particularly taxing and time consuming to perform.

U.S. Pat. No. 6,322,563 (“the '563 patent”), the content of which is incorporated by reference herein, describes a new and advantageous surgical fastener that can be used in furtherance of the ACI treatment, and in lieu of sutures. In accordance with the teachings of the '563 patent, a surgical fastener is retrieved from a holding area through the use of an applicator device, within which the fastener becomes retained via an interference fit. Using the applicator, the fastener is inserted into and through the membrane patch (i.e., periosteum), and into cartilage until the periosteum becomes held in place between the outer portion of the cartilage and a tail of the fastener. The applicator is then withdrawn, but the fastener remains anchored in place due to combination of the geometry of its proximal end and the holding force exerted by the cartilage. This fastening process is repeated as needed, i.e., until enough fasteners are in place to assuredly seal off the periosteum. The fastener(s) remain in place to graft the periosteum to the cartilage, after which the fastener(s) are bioabsorbed.

The process required to deploy the fasteners described in the '563 patent is quick, reliable, and does not involve the use of any unwieldy equipment. Thus, it represents a marked improvement as compared to the above-described suturing process, which requires far more time and effort in order to reach, at best, a similar end result.

However, it is possible that the fastener described in the '563 patent might not be ideally suited for the entire range of patients who undergo ACI treatment. For example, some ACI patients have severely damaged, non-robust cartilage, which, in turn, does not exert a great deal of anchoring/holding force.

Thus, in accordance with the teachings of the '563 patent, when one attempts to withdraw the surgical fastener applicator from the non-robust cartilage, it is possible that the strength of the existing interference fit between the fastener and the applicator will be greater than the anchoring/holding force exerted upon the fastener by the severely damaged cartilage. If that occurs, the fastener—instead of being retained within the cartilage—will remain in communication with the applicator, and the fastener insertion process will only result in a reamed, fastener-shaped bore being formed/defined through the patch and within the cartilage.

Moreover, the possibility of such unintended and undesired non-retention of the fastener within the target tissue can be increased due to side effects following sterilization of the fastener. For example, the fastener described in the '563 patent can be sterilized in ethylene oxide. Such sterilization could physically alter the geometry of (e.g., cause blunting of) the proximal end of the fastener and/or can create deformities in the body of the fastener. These problems (both individually or collectively) can cause a greater than anticipated interference fit between the fastener and the applicator, thus further elevating the risk that the fastener described in the '563 patent will not remain within the target tissue following its attempted insertion therein.

Accordingly, there is a need for improved surgical fastening devices, as well as methods for handling and inserting such devices, wherein the devices and their methods of insertion allow the devices to be quickly yet reliably applied/inserted—with little to no resulting trauma to a patient—into even severely damaged tissue/cartilage in order to provide small tissue approximation in situations that require multiple points of connection as well as fine precision.

### SUMMARY OF THE INVENTION

The present invention meets these, and other needs by providing novel surgical fastening devices, as well as methods for using such devices in connection with surgical or medical procedures, e.g., Autologous Chondrocyte Implantation (ACI).

The surgical fastener of the present invention is generally in the form of a tack, and comprises a conical head, a tail section, and an elongate (preferably flexible) rod extending between the conical head and the tail. The elongate rod and the conical head lie generally along the same longitudinal axis, and the elongate rod has a diameter less than the proximal diameter of the conical head. Thus, the back (proximal) surface of the conical head extends beyond and preferably is generally normal, or perpendicular to, the outer surface of the elongate rod.

The tail of the fastener extends radially from a proximal portion of the rod. A transverse locking member extends from the tail and also is offset from the longitudinal axis of the head and the rod. In preferred aspects of the present invention, the distal portion of the tail section of the fastener forms an acute angle with a back portion of the rod, and the transverse locking member is generally formed by a pair of shaped (e.g., cylindrical or frusto-cylindrical) protrusions that are located on each side of the tail section and that are offset from the axis of the head and rod members.

The preferred location of the transverse locking member (i.e., offset from the longitudinal axis of the head and the rod) is conducive to the positioning and maintenance of the tack in a desired location. Specifically, yet by way of non-limiting example, when the tack is used in holding a first object (such as a temporary covering) atop a second object (such as cartilage), the tack's head and rod are buried in the cartilage, and the temporary covering is lodged between the transverse locking member and the cartilage.

The back of the tack's conical head holds the tack in the cartilage and prevents unwanted motion of the tack backwards out of the cartilage. The transverse locking member holds the tack in the opposite direction and prevents the tack from moving forward through the temporary covering and into the cartilage.

The temporary covering is lodged in between the cartilage and the transverse locking member, such that the transverse locking member forms a seal between the cartilage and the covering. Specifically, the offset transverse locking member is designed to pull the covering upward under it, thereby permitting the tacked covering to form a seal between the two objects that is clean and flush or close to flush with the surface of the joined objects.

According to some exemplary aspects of the present invention, the surgical fastener further comprises a ramp-like support that extends proximally and inwardly from the back side of the conical head to the elongate rod, thereby further supporting the conical head.

Insertion of surgical fasteners of the present invention is aided by the use of one or more tack applicators. The tack applicators of the present invention preferably comprise an elongate handle, a loading tip, and a carrier assembly. More particularly, the carrier assembly generally comprises a body portion and a cannula extending from the front of the body portion. The cannula is provided with a slot on one side, from which the tail section, transverse locking member, and, preferably, the ramp-like support, all protrude. The conical head of the tack extends outwardly from and beyond the front of the cannula.

The outer diameter of the cannula is preferably sized to be less than or equal to the maximum diameter of the conical head of the tack, thereby minimizing the size of the insertion cut to be approximately equal to the size of the conical head of the tack. Preferably, the inner diameter of the cannula is sized to form a clearance fit with respect to the rod such that no portion of the rod is in contact with any portion of the inner diameter of the cannula.

The existence of a clearance fit between the cannula and the tack reduces—if not entirely eliminates—the possibility that the tack would be inadvertently (and disadvantageously) retained within the cannula following placement of the head of the tack within target tissue, even if that target tissue or cartilage is severely damaged so as to exert little retention force upon the tack.

To provide added assurance that the tack will be retained within the cannula prior to insertion of the tack within the target tissue/cartilage, fluid occupies predetermined portions along the tack. The presence of the fluid creates a meniscus, which, in turn, will create a capillary/retaining force between the cannula and the rod. The capillary force will be large enough to enable the tack to be retained within the cannula, but not so great as to prevent the tack from being deployed into target tissue, even if that tissue is excessively damaged and/or non-robust.

A wide variety of fluid(s) may be used to coat the predetermined area(s) of the tack; however the specific fluid chosen should be biocompatible, so as not to cause an adverse reaction in a patient, and should also be at least somewhat viscous, so as to promote the formation and maintenance of capillary induced cohesive force(s). The use of a hydrophobic fluid is currently preferred in order to ensure that the fluid remains stable (and, thus, to ensure that the capillary forces remain between the tack and the cannula) if the tack is treated with desiccants and/or is vacuum dried. Desiccants and vacuum drying processes are generally employed prior to packaging biodegradable medical devices (such as the tack described herein) in order to remove fluids (e.g., water) that can cause premature degradation of the devices, thus enabling the dried medical devices to enjoy a long shelf life prior to usage thereof. Despite the fact that the hydrophobic fluid will remain in place on the predetermined area(s) of the tack following any vacuum drying or desiccant use, and despite the belief within the art that the presence of fluid will cause premature degradation of a biodegradable article, observations made in furtherance of the development of the present invention have unexpectedly indicated that presence of the hydrophobic fluid on the predetermined area(s) of the tack did not appear to cause the tack to prematurely degrade.

The use of hydrophobic fluid to coat the predetermined area(s) of the tack also is preferred because it allows for the tack to be vacuum dried or treated with desiccants without a risk of the fluid disappearing or being reduced in quantity, thus, in turn, allowing for the fluid to remain present to create the meniscus that, in turn, creates the capillary induced cohesive forces for retaining the tack within the cannula.

In preferred aspects of the invention, at least one small protrusion, bump or other positive surface feature is defined/present on the outer surface of the cylindrical rod of the tack. The presence of the one or more protrusions will aid in the creation and maintenance of the capillary-induced retention force between the tack and the cannula.

The loading tip of the tack applicator is designed to grasp onto the body portion of the carrier assembly from the proximal end, with the conical head of the tack extending outwardly from and beyond the distal or front end of the loading tip. The loading tip may further include a means for providing added visibility to a user during tack delivery. For example, such visibility means may include a window or a notch along the loading tip.

The elongate handle of the tack applicator may be straight or curved, in part to provide optimal access to various bodily cavities. In addition, and in order to further aid in insertion of one or more tacks into tight areas, the tack applicator preferably tapers from the back end of the elongate handle towards the loading tip. To aid a user's grip on the handle, a portion thereof may have a textured surface, which prevents the applicator from slipping in one's hand.

Depending on the particular surgical procedure, the overall dimensions and shape of the tack applicator and carrier assembly may vary. For example, the tacks may be delivered either in an open procedure or arthroscopically, whereby arthroscopic procedures further involve the use of trocars or other hollow delivery mechanisms, through which the various tools required during the repair may access the site.

The overall design of the tack(s) and the tack applicator(s) is generally the same for both open and arthroscopic procedures; however, due to the nature of the latter procedures, a narrower and longer applicator assembly is generally required (i.e., for insertion through the hollow delivery mechanism). In aspects of the invention involving arthroscopic tack delivery, the tapered handle of the tack applicator and or the carrier assembly itself is/are longer and narrower than the corresponding equipment that is used in connection with open procedures.

The carrier assembly may further be made flexible along its length. For example, flexible plastic or kerfed material may be utilized to enable/facilitate insertion of the tacks through a curved hollow delivery mechanism. The carrier preferably is further designed to help maintain proper orientation of the tack so that, for example, the tack is not inadvertently inserted sideways or upside down. Thus, the carrier may have, for example, a rectangular or oval shaped cross section, or it may have a round or square cross section with a notch or raised area indicating the tack orientation.

To assist in holding and keeping track of tack and carrier assemblies, a cassette may be provided which holds a plurality of carriers, each holding a tack in position for use. The cassette generally comprises a number of parallel channels, each channel being sized to fit and hold a carrier assembly. Preferably, the entire cassette is disposable.

The channels of the cassette are preferably divided into more than one column. In such as aspect of the invention, two columns can be provided, wherein one column can hold new carrier assemblies with tacks loaded in the cannulas and the other column can be for placement of used carrier assemblies. Thus, after the tack is released from the cannula, the carrier assembly may be disposed of in the used carrier assembly disposal column. This not only aids in sanitary disposal of used carriers, but also facilitates the surgeon's ability to continually keep track of the number of tacks that have been utilized.

The channels of the cassette are preferably designed such that each tack's conical head is essentially at the front end of the channel, with a space provided/defined in front of the tack's head to protect the head and an additional space located/defined behind the back end of the carrier assembly. The channels are sized such that the tack applicator slides into the space behind the back end of the carrier assembly and into alignment with the carrier assembly, thereby ensuring proper loading of the carrier apparatus into the loading tip of the tack applicator. In accordance an exemplary aspect of the present invention, there may be guides on the sides of each channel to further aid in proper insertion of the tack applicator.

The tack(s) and tack applicator(s) are generally used as follows: a tack is first inserted into the cannula of the carrier apparatus, tail end first, thus forming the clearance fit and the carrier-induced capillary retention force described above. The tail, ramp-like support and transverse locking member of the tack each extend upwards out of the corresponding slot in the cannula. The sharp, conical head of the tack extends out and beyond the front of the carrier apparatus. The carrier assembly is then mounted into the loading tip of the tack applicator such that the back end of the carrier assembly is inserted first and the conical head of the tack extends out and beyond the entire tack applicator assembly.

In the case where a cassette is used, the carrier assemblies with loaded tacks are preferably pre-mounted into one column of cassette channels. This enables the tack applicator to be simply pushed into a channel, with its loading tip first, until the loading tip engages and locks onto a carrier assembly. The tack applicator is then withdrawn from the channel, with a carrier assembly mounted therein.

The tack is now ready for insertion into a desired site, wherein insertion is generally accomplished as follows: the tack applicator is placed at the point of insertion with the sharp conical head of the tack leading the way. The tack applicator is pushed forward into the tissue as the sharp, conical head penetrates through the tissue.

Once the tack has been pushed to the proper depth within the tissue, the tack applicator is gradually pulled out of the tissue. Dulling this pulling, the tack's tail, transverse locking member, and back end of the conical head hold the tack securely in place within and in contact with the tissue. This will cause the tissue (even if damaged or non-robust) to exert enough force to counteract and overcome the capillary-induced retention force that had been defined between the tack and the cannula, thus causing disengagement of the tack from the cannula of the carrier assembly, and preventing the tack from backing out of the tissue as the cannula is withdrawn from the tissue. Thus, the tack remains lodged within the tissue as the tack applicator is withdrawn.

Where the tack is used to repair cartilage and/or bone, the site is first prepared by methods known in the art. For example, one such method calls for excising all damaged or unhealthy cartilage from the perimeter of the defect. The size of the defect is then measured and a patch of natural or synthetic membrane, such as a periosteal patch, of appropriate size is placed over the defect.

One or more of the tacks of the present invention are then used to secure the patch over the defect by the tack insertion procedure outlined above. The use of small sized tacks is essential in such applications because the site being repaired is particularly thin and the cartilage at the site is often relatively soft.

Attachment of the periosteal patch is accomplished by inserting tacks about the outer perimeter of the patch and close to its edges. The tack must not create too large of a hole, as that may pull and tear through the edge of the patch. Thus, the use of conventional (i.e., larger) tacks is not a viable option since such tacks will not fit within the site and could potentially tear through the edges of the thin and delicate periosteum. Moreover, conventional tacks are inappropriate for the periosteum and the like tissue because they are rigid and resist bending, and thus would cause additional stress and pain to the area.

More specifically, fasteners, applicators and methods of the present invention are particularly suited for use in connection with the repair of damaged cartilage using an Autologous Chondrocyte Implantation (ACI) treatment, which is discussed, for example, in Brittberg et al, “Treatment of Deep Cartilage Defects in the Knee With Autologous Chondrocyte Transplantation”, New England Journal of Medicine, 331:889-895 (Oct. 6, 1994) and Minas et al, “Chondrocyte Transplantation”, Operative Techniques in Orthopaedics, Vol. 7, No. 4, pp. 323-333 (October 1997)).

The ACI process/treatment is advantageous in that it restores the articular surface of cartilage (e.g., knee cartilage), without compromising the integrity of healthy tissue or the subchondral bone. ACI treatment generally involves the following procedure: entering the knee to remove a biopsy of healthy cartilage tissue, which may then be cultured externally. Thereafter, the knee is again entered and the defect site is prepared by removing the damaged cartilage and measuring the lesion size.

The cultured cartilage is then placed into the lesion and sealed off with a natural or synthetic membrane patch (e.g., a periosteum patch), using one or more tacks of the present invention. Within the lesion, the cells continue to multiply and integrate with surrounding cartilage. Over time, the cells continue to mature and fill in the lesion with healthy cartilage.

Defects that qualify for ACI treatment generally comprise a lesion surrounded by healthy cartilage, often called a focal chondral. Such defects involve relatively soft and thin cartilage. Further, the temporary patch utilized in such procedures is thin and delicate; therefore, the use of small sized tacks such as those of the present invention is essential in such applications. Still further, attachment of the temporary patch is accomplished by inserting fasteners about the outer perimeter of the patch close to the edges of the patch. Thus, the small tack of the present invention is beneficial, because it does not create too large of a hole, which would pull and tear through the edge of the patch.

Conventional tacks are not suitable for such delicate procedures as ACI due to their rigid, large structures. Such tacks, instead, are designed for adhering together two pieces of tough meniscal cartilage by inserting only a small number of large meniscal tacks through the central area of the meniscal cartilage. Typically the two pieces of meniscal cartilage being fixed together are similar in size, and, thus, the meniscal tacks must be large enough so that the tack extends through both pieces of the meniscus.

### BRIEF DESCRIPTION OF THE DRAWINGS

FIG. 1 shows a side view of a surgical tack in accordance with one embodiment of the present invention.

FIG. 2 shows a perspective view of the surgical tack shown in FIG. 1.

FIG. 3 shows a front view of the surgical tack of FIG. 1.

FIG. 4 shows a top view of the surgical tack of FIG. 1.

FIG. 5 shows a back perspective view of the surgical tack of FIG. 1.

FIG. 6 shows one embodiment of a tack applicator in accordance with the present invention.

FIG. 7 shows a second embodiment of a tack applicator in accordance with the present invention.

FIG. 8 shows a perspective view of a carrier cassette assembly holding a number of carriers provided with surgical tacks ready to be picked up by a tack applicator and used in accordance with the present invention.

FIG. 8b shows a perspective view of a carrier cassette assembly in accordance with the present invention.

FIG. 9 shows an enlarged view of one embodiment of a carrier assembly in accordance with one embodiment of the present invention.

FIG. 9a shows an enlarged view of one embodiment of a carrier assembly holding a tack in accordance with the present invention.

FIG. 9b shows an enlarged, top view with cut away of FIG. 9a with the tack being maintained within the cannula of the carrier assembly via a clearance fit.

FIG. 9c is an enlarged view of the clearance fit of FIG. 9b.

FIG. 10 shows the tack applicator being inserted into the cassette for carrier assembly loading.

FIG. 11 shows an enlarged view of FIG. 10.

FIG. 12 shows a curved handled tack applicator and the cassette for carrier assembly loading.

FIG. 13 shows a cassette and a curved handled tack applicator with a loaded carrier assembly.

FIG. 14 shows the surgical tack of FIG. 1 being used to repair cartilage by use of a natural or synthetic membrane patch.

FIG. 15 shows a view of the portal sites to the right knee.

FIG. 16 shows one type of arrangement of trocars or hollow delivery tubes for use in arthroscopic procedures on the right knee.

FIG. 17 shows one method of preparing a natural or synthetic membrane patch for arthroscopic insertion.

FIG. 18 shows the natural or synthetic membrane patch as prepared by FIG. 18 being inserted arthroscopically.

FIG. 19 shows a natural or synthetic membrane patch being fixed to a site arthroscopically.

FIG. 20 shows excess patch being trimmed from the fixed patch arthroscopically.

FIG. 21 shows various carrier assembly embodiments in accordance with the present invention as used in arthroscopic procedures.

FIG. 22 shows a top view of one embodiment of a spring loaded tack applicator of the present invention.

FIG. 23 shows a side view of the tack applicator of FIG. 22.

FIG. 24 shows a top view of a second embodiment of a spring loaded tack applicator of the present invention.

FIG. 25 shows a side view of the tack applicator of FIG. 24.

FIG. 26 shows an isometric view of one embodiment of the tack applicator tip having a window in accordance with the present invention.

FIG. 27 shows an isometric view of one embodiment of the tack applicator tip having a notch in accordance with the present invention

FIG. 28 shows an isometric view of a second embodiment of the tack-applicator tip having a notch in accordance with the present invention

FIG. 29 shows one embodiment of the spring assembly as mounted within a straight handled tack applicator and one embodiment of the spring assembly as mounted within a curved handled tack applicator in accordance with the present invention.

### DETAILED DESCRIPTION OF THE INVENTION

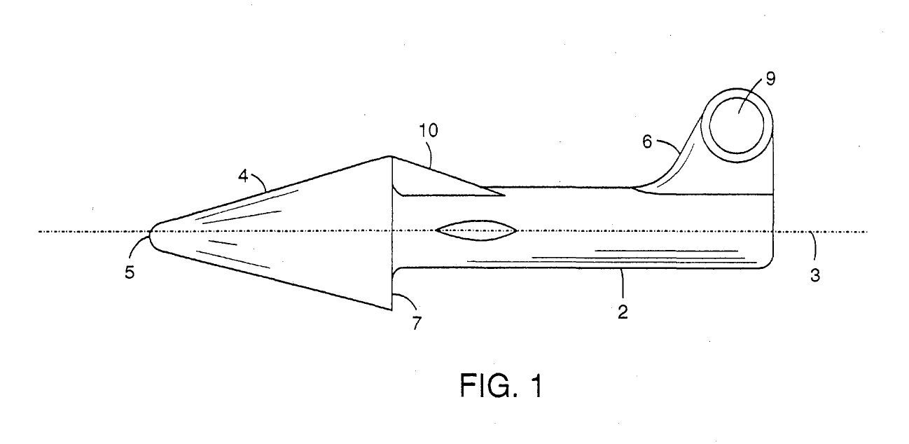

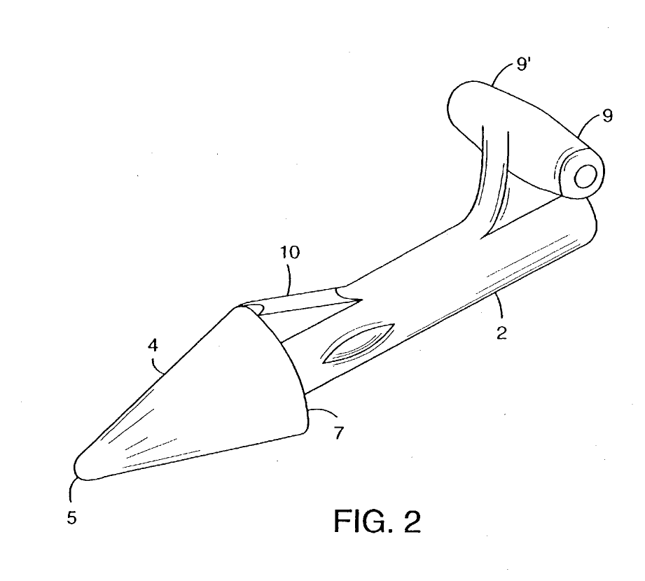

Referring now to the various figures of the drawings, wherein like reference characters refer to like parts, there is shown in FIGS. 1-5 various views of a surgical tack 1 in accordance with the present invention.

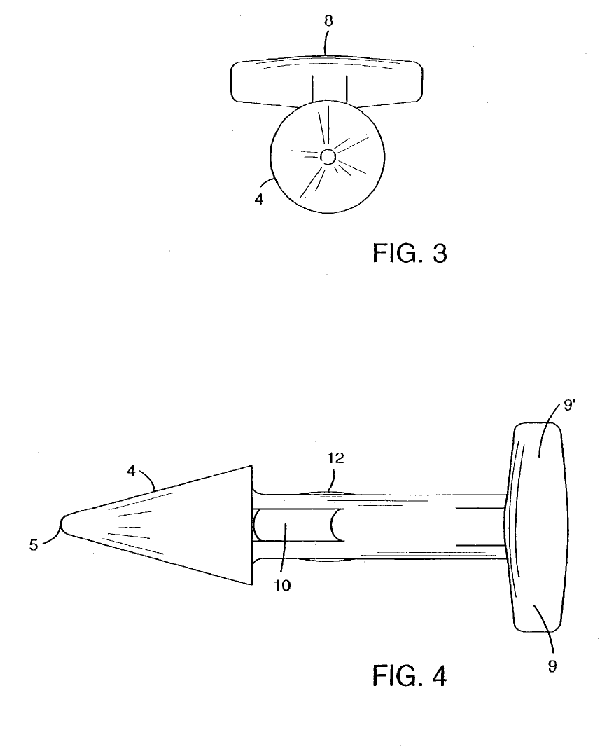

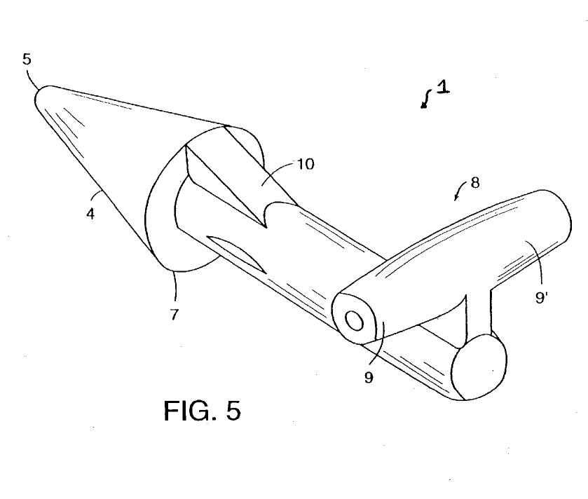

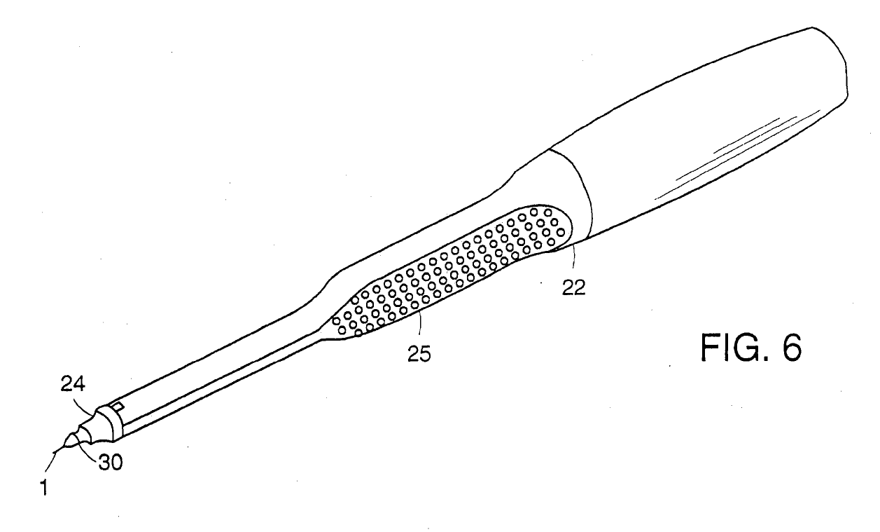

The tack 1 comprises a generally cylindrical rod 2, having at its distal end a sharp conical head 4 and at its proximal end a tail 6. The tack 1 also may include one or more protruding bumps 12 (see, e.g., FIG. 4), which assist in retaining the tack within a delivery device (e.g., a carrier assembly) as will be described below. The distal tip of the head 4 is sharp, having a minimum manufacturable radius of curvature so that the head can easily penetrate target tissue. Preferably, but not necessarily, the head 4 and the rod 2 share substantially the same longitudinal axis 3. The tack tail 6 preferably extends proximally and axially to the back end of the rod 2, and also preferably extends from the rod to form a substantial angle with the longitudinal axis 3. As shown in FIGS. 3 and 5, a transverse, locking member 8 extends from the tail 6 of the tack 1, is offset from the longitudinal axis 3, and is spaced from the surface of the rod 2. Preferably, the locking member 8 is formed by two generally symmetrical protrusions 9 and 9′ (see FIG. 4) that extend, respectively, from opposite sides of the tail 6, each in a direction transverse the longitudinal axis 3.

In a preferred embodiment of the present invention, the protrusions 9, 9′ are frustro-cylindrical in shape and are rounded at their outermost extremities. A locking member 8 with such protrusions 9, 9′ extending transverse to the longitudinal axis of the rod 2 and head 4, is capable of effectively securing delicate and thin tissue, such as periosteal tissue, and of preventing the secured tissue from slipping through the tack 1 and over the locking member 8. Moreover, the smooth surface of the frustro-cylindrical protrusions 9, 9′ also is advantageous in that it is effective to minimize the potential for tearing of the secured tissue, even thin, delicate tissue such as periosteal tissue. The protrusions 9, 9′ may be of generally constant or varying diameter. By way of non-limiting example, and as shown in FIG. 4, the protrusions 9, 9′ may decrease in diameter from the tail 6 towards their outermost extremities.

The sharp conical head 4 of the tack 1 also has a back end 7, which, as depicted in FIGS. 1, 2 and 5, is substantially flat and generally normal to the rod 2. The conical head 4 of the tack 1 is sized such that its back end 7 has a diameter greater than that of the rod 2, thereby allowing the back end to aid in anchoring the tack 1 in place within tissue and membrane upon delivery, as will be described in more detail below. The juncture between the back end 7 of the conical head 4 and the rod 2 may be at a generally sharp angle (e.g., a substantially perpendicular angle), or, as shown in FIGS. 1 and 5; the connection may form a smooth and slightly concave curve or fillet.

The tack 1 also preferably includes a ramp-shaped support 10 that extends from the back end 7 of the conical head 4 and that connects to the rod/body 2 of the tack, thereby providing further stability and support for the conical head 4. Preferably, the ramp-shaped support 10 is a solid member that extends from the outermost diameter of the back end 7 of the conical head 4. As shown in FIG. 5, the ramp-shaped support 10 may have substantially angular edges or, alternatively, may have slightly rounded edges.

The tack 1 preferably has a maximum length of approximately 0.25 inch, more preferably a maximum length of about 0.2 inch, and even more preferably, a length in the range of about 0.12 inch to 0.16 inch. The rod 2 preferably has a maximum diameter of about 0.03 inch, more preferably about 0.02 inch, and even more preferably in the range of about 0.015 inch to 0.017 inch, not taking into account the measurement(s) of any element(s) that protrude(s) from or is/are in communication with the rod. The maximum diameter of the head 4 is preferably about 0.05 inch, more preferably about 0.04 inch, and even more preferably in the range of about 0.029 inch to 0.033 inch.

In a currently preferred embodiment of the present invention, the following combination of dimensions are used: a rod length of between about 0.12 inch and 0.13 inch, a maximum rod diameter of between about 0.015 inch and 0.017 inch, and a maximum cone diameter of between about 0.029 inch and 0.033 inch.

Preferably, the maximum diameter of the rod 2 is approximately one-half the maximum diameter of the head/cone 4. Thus, as the rod 2 increases in size, the head 4 increases in size proportionally, and preferably, all other dimensions also increase proportionally as well. In an alternative preferred embodiment, the dimensions of the cone 4 are enlarged by up to 50% while keeping all other dimensions of the tack 1 substantially constant.

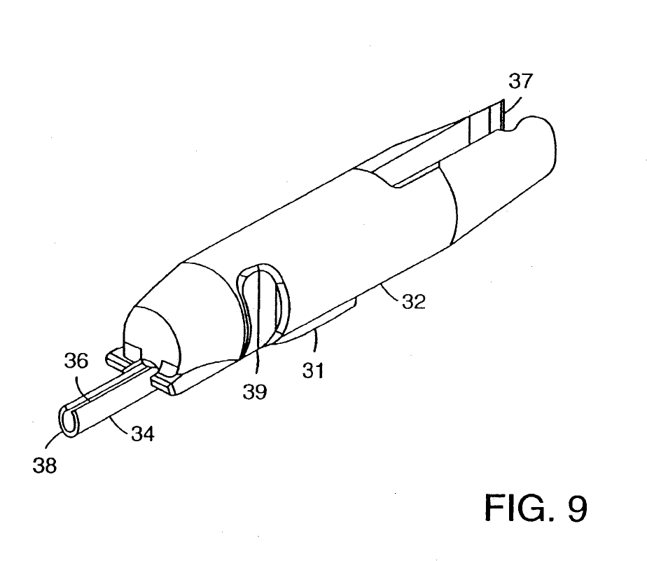

As shown in FIGS. 9A-9C, the tack 1 may further contain at least one positive surface feature 12 (e.g., a bump, ramp or other protrusion) that is located/defined on the rod 2 and that further aids in retaining the tack 1 within a cannula 34 (e.g., via a clearance fit), as will be described in further detail below. Although two protrusions are depicted in each of FIGS. 4 and 9B, the number of protrusions can be less than or greater than two in accordance with the present invention.

In an embodiment where there is more than one protrusion 12, each protrusion can protrude from the rod 2 by an identical or different amount. Each protrusion(s) 12 preferably (but not necessarily) protrudes from the rod 2 by a maximum distance of about 0.004 inch or less, more preferably by maximum distance of about 0.003 inch or less, even more preferably by a maximum distance in the range of about 0.0015 inch to 0.0025 inch, and currently most preferably by a maximum distance of about 0.0020 inch. Preferably, the maximum distance of protrusion of the surface feature(s) 12 from the rod 2 is approximately equal to one tenth to one fourteenth of the maximum rod diameter.

The surgical tack 1 is preferably formed of a bioabsorbable material. Suitable bioabsorbable materials for forming the tack 1 of the present invention include, but are not limited to, polyglycolic acid, polylactic acid, polydioxanone, polycaprolactone, trimethyl carbonate and copolymers thereof.

As will be explained in more detail below, an applicator is used to hold the rod 2 of the tack 1 rigid during insertion. Thus, the tack 1 may be formed from less rigid, more rapidly degrading bioabsorbable materials—that is, a more comfortable and flexible tack (one which does not resist bending, and which is absorbed more quickly by the body) may be utilized.

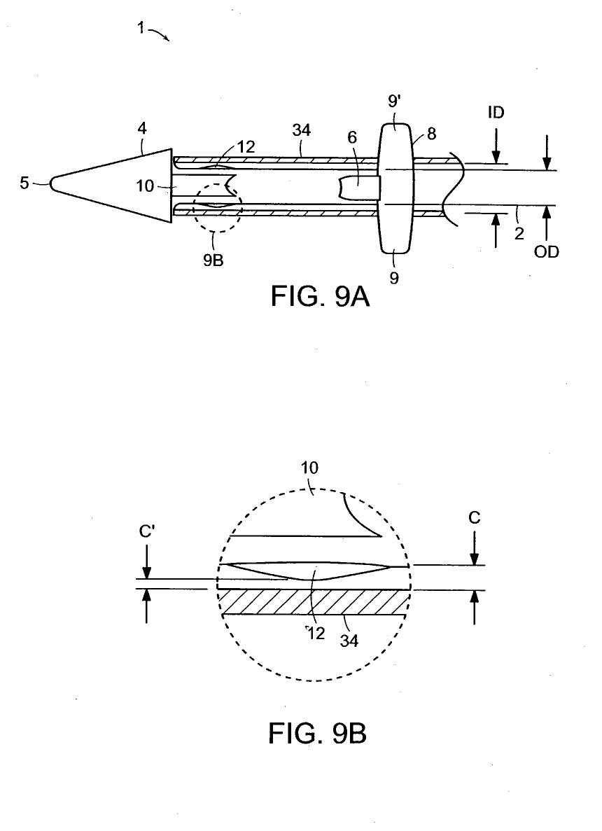

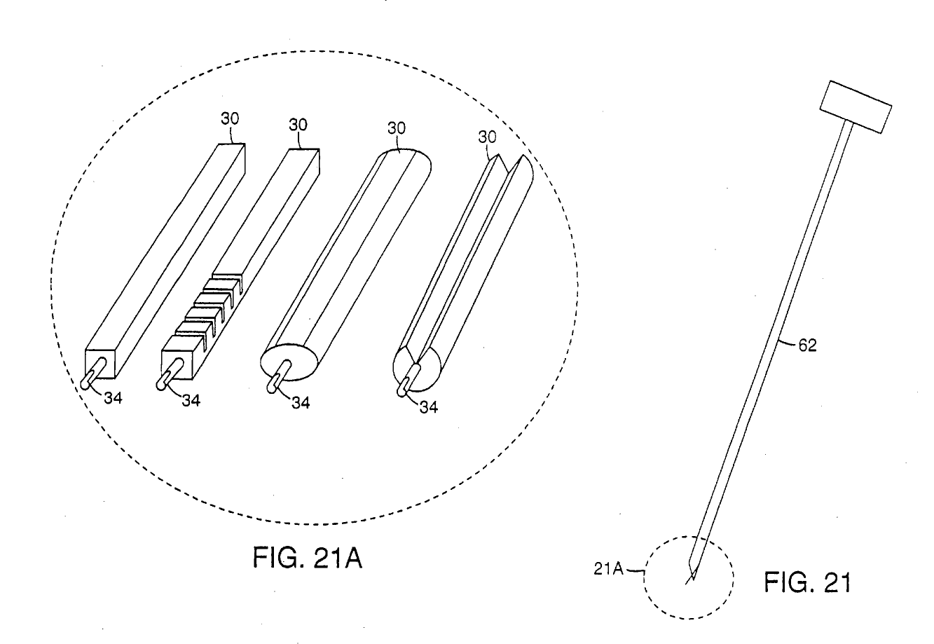

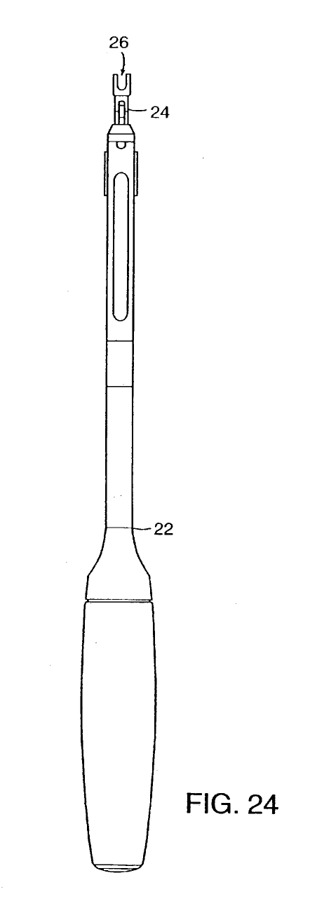

FIGS. 10-13 and 22-25, depict various views of tack applicators 20 in accordance with the present invention. The use of tack applicators 20 aids in holding a tack 1 and in allowing the tack to be properly aligned and oriented during placement of the tack into a target tissue site.

The tack applicators 20 comprise, in general, an elongate handle portion 22 and a loading tip 24. The handle portion 22 is designed to provide an individual with a comfortable and steady grip, and is preferably an elongate member.

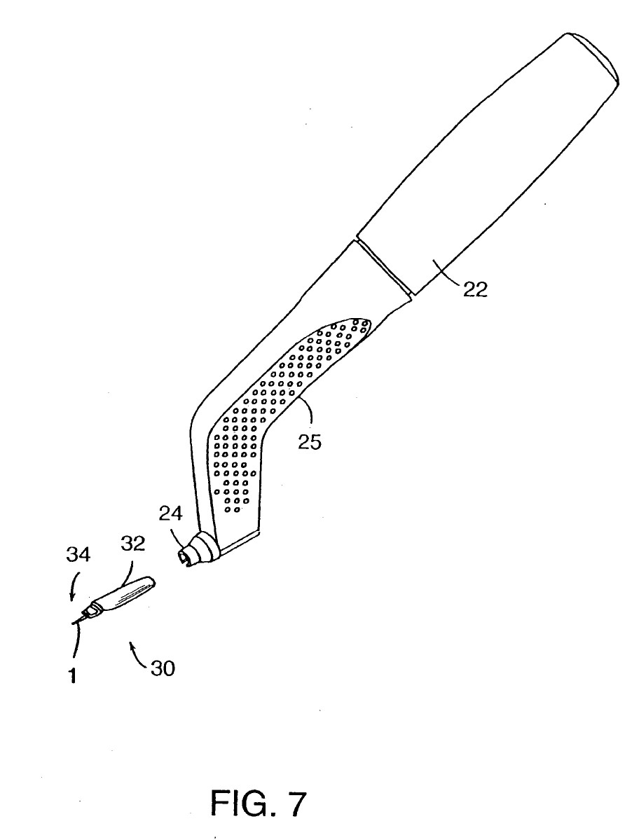

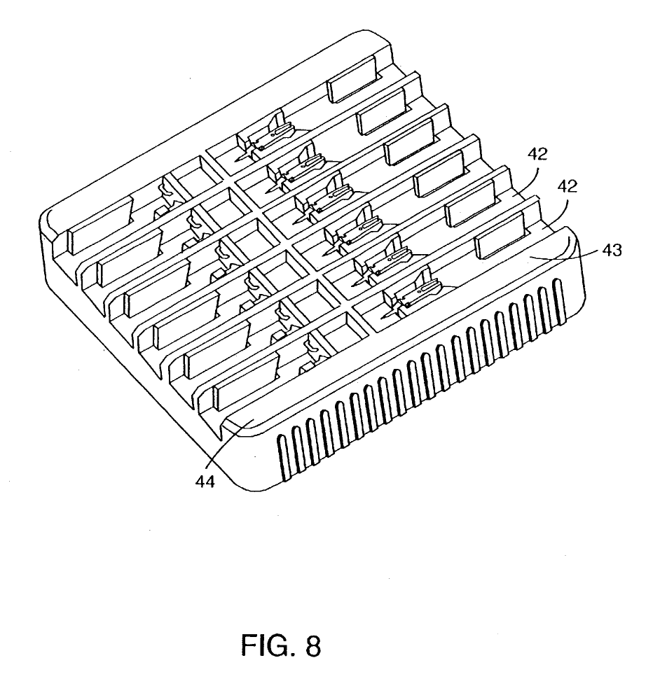

The handle may be substantially straight (as shown in FIGS. 6, 10 and 11) or it may be at least partially curved (as shown in FIGS. 7, 12 and 13), with the curved design providing—at least in certain instances of usage—enhanced accessibility into certain areas of the body. As indicated in the figures, the handle 22 of the applicator 20 can (and preferably does) taper towards the loading tip 24 to provide better access into tight areas.

No specific handle shape is currently preferred when using the applicator 20 in connection with an ACI procedure.

In accordance with the present invention, and as shown in FIGS. 6, 7, 10, 12 and 13, a textured surface 25 may be defined/included along the handle 22 towards the loading tip 24 in order to allow a user to obtain/maintain an enhanced grip upon the tack applicator 20. During use of an applicator 20 that includes the textured surface 25, the upper portion of the handle 22 generally would be enclosed in a user's palm, while the user's fingers—particularly the user's thumb and index finger—would be able to rest on the textured surface 25.

As shown in FIGS. 6 and 7, a carrier assembly 30 is removably mounted in the loading tip 24 of the applicator. The carrier assembly 30 assists in handling and properly aligning the small sized tacks 1 within the tack applicator 20, and comprises, in general, a body portion 32 and a cannula 34.

The cannula 34 extends from the front of the body portion 32 of the carrier assembly 30, and is the portion of the carrier assembly that enables the tack 1 to be maintained within the carrier assembly after the tack is loaded within the carrier assembly and until the tack is deployed into the target tissue. The cannula 34 is provided with at least one slot 36 (see, e.g., FIG. 9) that is shaped to allow for the support 10, the tail 6, and/or the transverse locking member 8 of the tack 1 to be received within the cannula.

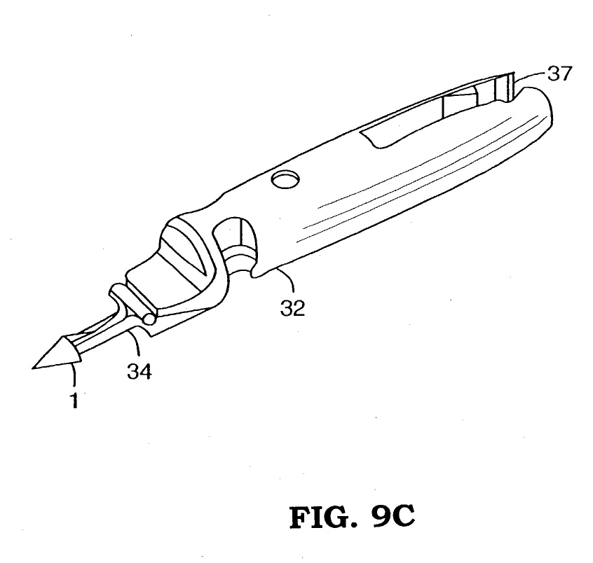

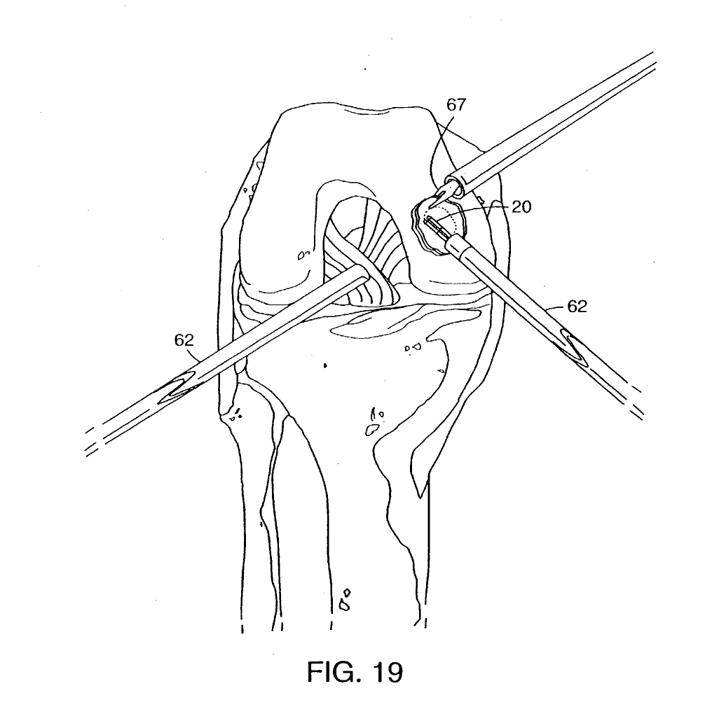

The tack 1 enters the cannula 34—tail 6 first—via a cannula opening 38. As shown in FIG. 9, the conical head 4 of the tack 1 protrudes from the opening 38 following insertion of the tack into the cannula 34. This arrangement facilitates the ability of the tack 1 to be deployed into target tissue as will be explained in detail below. In an exemplary embodiment of the present invention (and as shown in FIGS. 9 and 11), the cannula 34 has an overall cylindrical shape and a circular cross section. However, the cannula 34 may alternatively have a square, rectangular or oval cross section, e.g., as shown in FIG. 21. Preferably, the inside of the cannula is cylindrical in order to correspond to the tack's generally cylindrically shaped rod 2.

The fit that is maintained between the tack 1 and the cannula may be an interference fit, a friction fit or other types of fit. However, in a currently preferred embodiment of the present invention, the tack 1 is maintained within the tack via a clearance fit, such that no portion of the rod 2 is in contact with any portion of the inner diameter of the cannula. The existence of a clearance fit between the cannula 34 and the tack 1 reduces—if not entirely eliminates—the possibility that the tack will be inadvertently (and disadvantageously) retained within the cannula during withdrawal of the applicator 20 following placement of the head 4 of the tack 1 within target tissue, even if that target tissue or cartilage is severely damaged so as to exert little retention force upon the tack.

In an exemplary embodiment of this invention (see FIGS. 9b and 9c), the inner diameter, ID, of the cannula 34 is in the range of about 0.0195 inch to about 0.0210 inch, and the outer diameter, OD, of the rod 2 is in the range of about 0.0150 inch to about 0.0170 inch, except at the protruding area(s) 12, where the outer diameter of the rod increases by a maximum of about 0.002 inch. Thus, the amount of clearance, C, between the rod 2 and the inner diameter of the cannula 34 is in the range of about 0.0025 to 0.006 inch, preferably about 0.003 inch, except at the protruding area(s) 12 of the rod, where the amount of clearance, C′, is about 0.0005 to 0.004 inch, preferably 0.001 inch (i.e., the presence of the at least one surface feature reduces the amount of clearance at the at least one surface feature by approximately 66%).

Although the clearance fit allows for the tack 1 to more reliably be retained—upon insertion—within target tissue/cartilage, it also provides somewhat less assurance that the tack 1 will be retained within the cannula prior to insertion of the tack 1 within the target tissue/cartilage. To provide added assurance that the tack 1 will be, in fact, retained within the cannula prior to insertion of the tack within the target tissue/cartilage, fluid is introduced onto predetermined portions of the tack 1. Preferably, fluid is introduced onto at least one (preferably all) of the protruding area(s) 12 of the tack 1, but may be introduced onto other areas of the tack as well.

The presence of the fluid creates a meniscus, e.g., at the protruding area(s) 12 of the tack. Because of the small amount of clearance between the rod 2 and the cannula 34—especially the very small amount of clearance between the protruding area(s) 12 of the rod and the cannula 34—the meniscus will create a capillary/retaining force between certain areas of the cannula 34 and the rod, including between one or more of the protruding area(s) of the rod and the cannula.

Due to the nature of the capillary force that is exerted by a meniscus, the resulting capillary force will be large enough to enable the tack 1 to be retained within the cannula (and, thus, within the applicator 20 with which the cannula is in communication), but not so great as to prevent the tack from being deployed into target tissue and maintained—following deployment—within the target tissue, even if that tissue is damaged to an extent that it exerts very little holding force upon the tack. As noted above, this is highly advantageous, and addresses a potential area of concern with respect to the tissue fixation system of the U.S. Pat. No. 6,322,563 patent.

The fluid can be introduced onto the predetermined area(s) of the tack 1 by techniques known in the art. By way of non-limiting example, the fluid can be introduced onto the predetermined area(s) of the tack through the use of an applicator (e.g., a brush-type applicator or a spray-type applicator) or by directly introducing (e.g., dipping) the predetermined area(s) of the tack into a containment element (e.g., a receptacle or holder) that contains fluid.

Once the fluid has been introduced onto the tack 1 as desired, the tack can be inserted into the cannula 34. The tack 1 should be inserted so as to enable the meniscus to be created and the associated capillary induced cohesive force to be exerted. This force should be large enough to retain the tack 1 within the cannula 34 despite the vibrations and jostling encountered during manufacturing, sterilization, packaging, shipping, storage, and unpackaging of the equipment.

A wide variety of fluid(s) may be used to coat the predetermined area(s) of the tack 1; however the specific fluid chosen should be biocompatible, so as not to cause an adverse reaction in a patient, and should also be at least somewhat viscous, so as to promote the capillary induced cohesive force. Preferably, the fluid is a hydrophobic fluid; exemplary hydrophobic fluids include, but are not limited to, Dow Corning Fluorosilicone F-1265 and Dow Polyglycol P-2000.

The use of a hydrophobic fluid is currently preferred in order to ensure that the fluid remains stable (and, thus, to ensure that the capillary forces remain between the tack 1 and the cannula 34) if the tack 1 is treated with desiccants and/or is vacuum dried. Desiccants and/or vacuum drying processes are generally utilized prior to packaging biodegradable medical devices (such as the tack 1 described herein) in order to remove fluids (e.g., water) that can cause premature degradation of the devices, thus enabling the dried medical devices to enjoy a long shelf life prior to usage thereof.

Despite the fact that the hydrophobic fluid will remain in place on the predetermined area(s) of the tack 1 following any vacuum drying or desiccant use, and despite the belief within the art that the presence of fluid will cause premature degradation of a biodegradable article, observations made in furtherance of the development of the present invention unexpectedly indicate that presence of the hydrophobic fluid on the predetermined area(s) of the tack 1 do not cause the tack to prematurely degrade.

Exemplary sterilization techniques that are applicable to the tack 1 of the present invention include, but are not limited to, gamma or e-beam sterilization. Such sterilization techniques are currently preferred because they are not performed in an environment and/or under conditions that could cause modification of the geometry (i.e., edge blunting and/or shape changes) of the tack 1.

The use of hydrophobic fluid to coat the predetermined area(s) of the tack 1 also is preferred because it allows for the tack to be vacuum dried or treated with desiccants without the fluid disappearing or being reduced in quantity. Thus, the fluid remains present on the predetermined area(s) of the tack 1 to create a meniscus, which, in turn, creates capillary induced cohesive forces between the tack and the cannula 34 that will act to retain the tack within the cannula prior to insertion of the tack within target tissue.

In accordance with the present invention, it is possible to load the tack 1 within the cannula 34 after the carrier assembly 30 has been loaded into the tack application 20; however, it is currently preferred to load the carrier assembly into the tack applicator after the tack 1 has been loaded into the cannula 34 of the carrier assembly.

In furtherance of the carrier assembly 30 being loaded into the tack applicator 20, the loading tip 24 (see FIG. 7) of the tack applicator grasps and locks onto the back of the body portion 32 of the carrier assembly 30. The body portion 32 may fit within the loading tip 24 by any means known in the art, including—by way of non-limiting example and as illustrated in FIG. 9-a frictional fit, which may be aided by at least one groove 37 in the back of the carrier assembly body portion 32 that corresponds to a protrusion (not shown) within the loading tip 24. As the carrier assembly 30 is being loaded into the tack applicator 20, the loading tip 24 fits and locks within the groove 37.

As shown in FIG. 9, there may further be a protrusion 31 along the body 32 of the carrier assembly 30. That protrusion 31 fits within a corresponding slot (not shown) in the loading tip 24 to aid in proper alignment of the carrier 30 within the loading tip. Such an arrangement also helps to prevent unwanted rotation of the carrier assembly 30 within the loading tip 24.

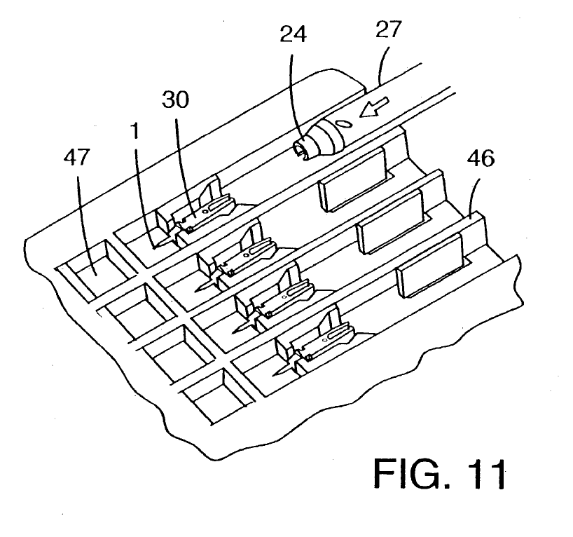

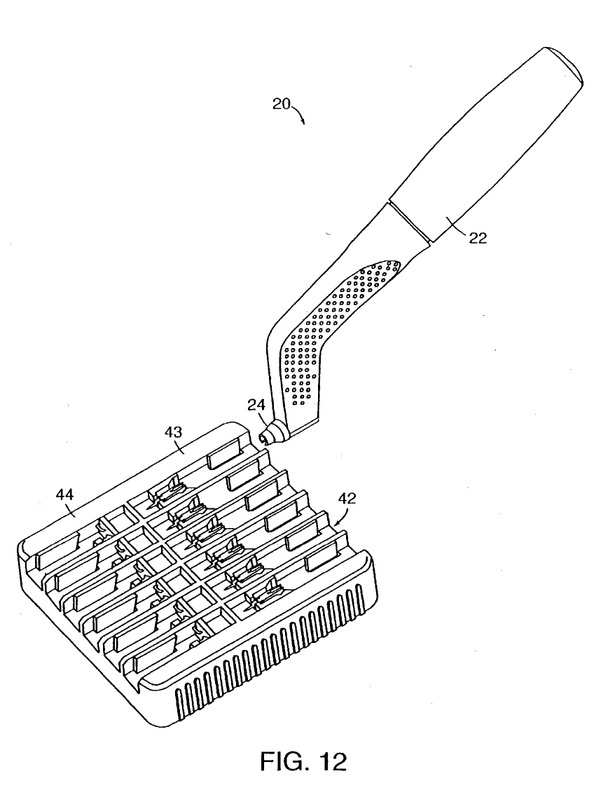

The carrier assembly 32 is preferably inserted within the loading tip 24 of the tack applicator 20 such that a substantial part of the carrier body portion 32 is housed within the loading tip 24 following the insertion process, since such an arrangement provides increased hold and promotes the stability of the carrier assembly within the tack applicator. However, as shown in FIG. 13, the insertion process should allow the cannula 34 and the tack 1 to extend out the front of the loading tip 24 and beyond the front of the tack applicator 20, such that the tack can be deployed into the target tissue as shown in FIG. 14.

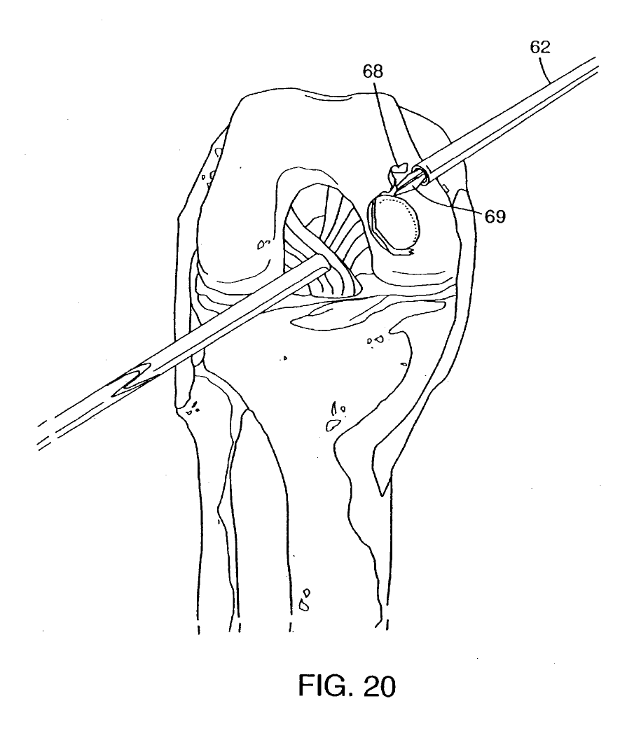

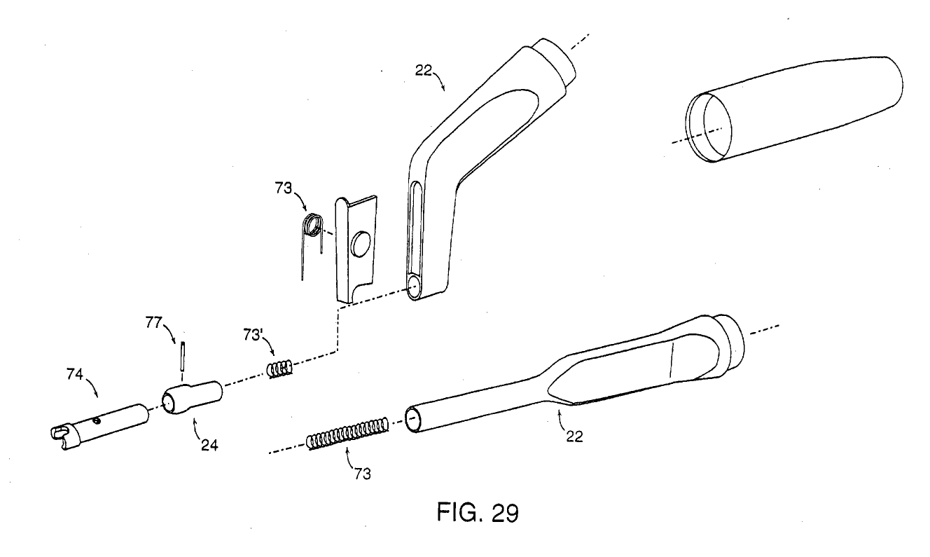

In some applications, a tack viewing means 26 (see FIGS. 22, 24 and 26-28) and a spring loading mechanism 73 and 73′ (see FIG. 29) may be incorporated as part of the tack applicator 20 in order to provide a user with a view of the tack being inserted and to aid in controlling the depth of insertion of the tack 1 within the target tissue. The tack 1, when loaded within the tack applicator 20, will be located below the viewing area 26. And as the tack is inserted into target tissue, the tack 1 will pass across and under the viewing area to allow for visualization of the tack.

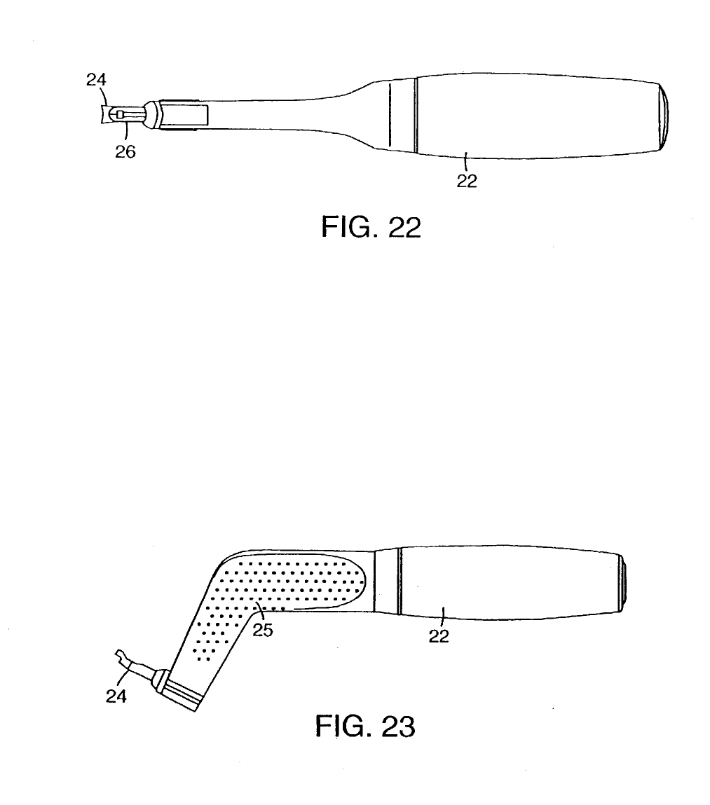

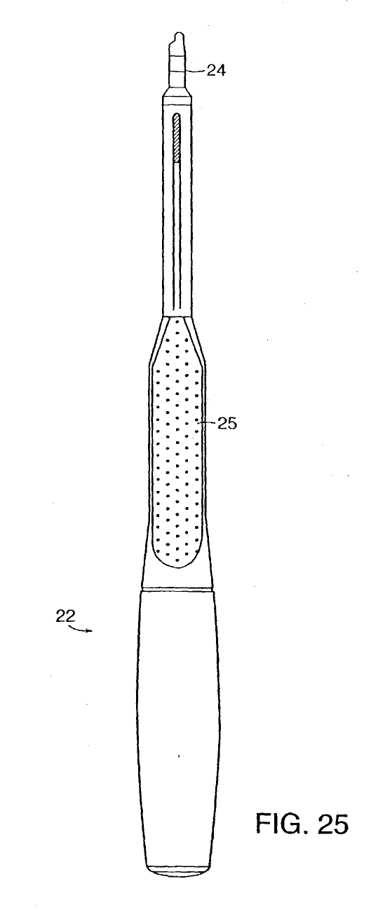

The viewing means/area 26 of the tack applicator 20 may be, for example, a window or notch (as shown in FIGS. 22 and 24) and is preferably located within a guide mechanism 74 that itself is preferably included in the spring loaded tack applicator, as illustrated in FIG. 29. An enlarged visual depiction of the viewing means 26 in the guide mechanism 74 is shown in FIGS. 26-28.

Preferably, and as depicted in FIG. 26, the guide mechanism 74 is designed with a top extension 70 and a bottom bumper portion 71 for seating against the exterior surface of target tissue as the tack 1 is inserted therein. In accordance with an embodiment wherein an Autologous Chondrocyte Implantation (ACI) treatment to repair knee cartilage is being performed, the bumper portion 71 is effective to securely hold the periosteal patch against the knee cartilage as the tack 1 is being inserted into the cartilage.

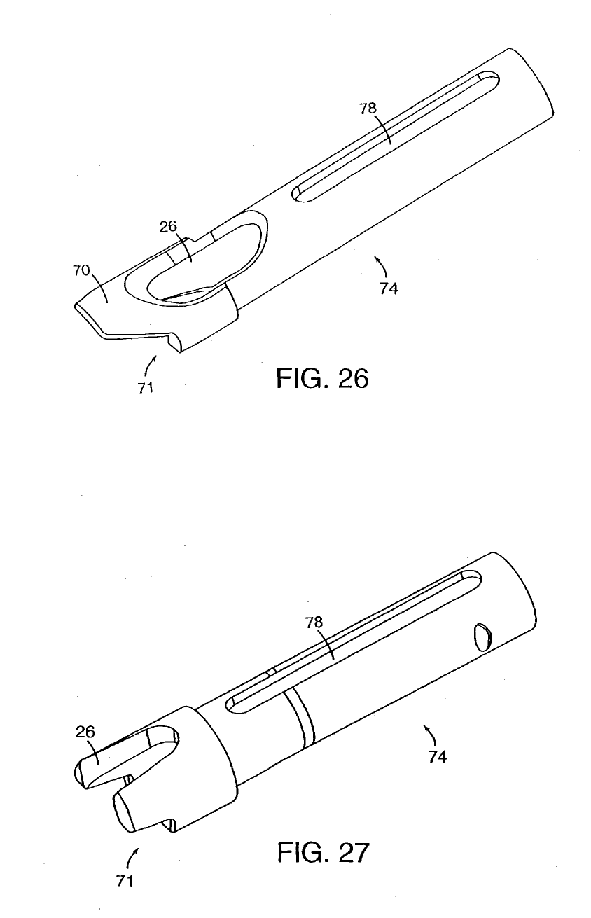

In another exemplary embodiment of the present invention, and as shown in FIG. 27, a notch 26 may be located within the guide mechanism 74. The notch 26 is useful, e.g., in that a holding tool, such as forceps (not shown), may be inserted through the notch 26 to hold the periosteal patch steady as the tack 1 is being inserted therein. The bottom bumper portion 71 of the guide mechanism 74 may also be included in this embodiment.

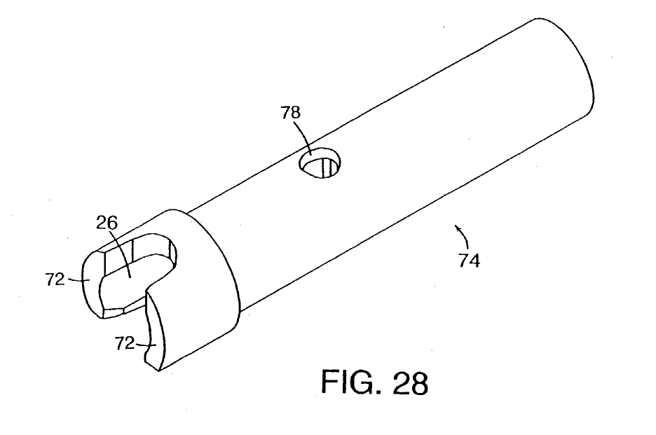

FIG. 28 depicts yet another embodiment in which a notch 26 is located in the guide mechanism 74. Again, a holding tool, such as forceps (not shown), may be inserted through the notch 26 to hold the periosteal patch steady as the tack 1 is being inserted therein. In this embodiment, however, additional bumper portions 72 may be provided for seating against the exterior surface of the target tissue as the tack 1 is being inserted, in order to prevent the applicator 20 from entering the target tissue.

To ensure proper depth of insertion and to aid in gradual dislodgement of the tack 1 from within the cannula 34 as the applicator 20 is withdrawn from the site, a spring mechanism may be present within the applicator. The spring mechanism is preferably designed such that a spring 73 is fully extended once the tack 1 is loaded in the loading tip 24. Upon pushing the tack 1 into the target tissue, the spring 73 compresses until it is fully compressed, wherein detent spring 73′ prevents further movement of the guide mechanism 74. Once the spring 73 is fully compressed, the bottom bumper portion 71 of the guide mechanism 74 maintains the applicator 20 external to the target tissue and prevents further pushing of the tack 1 into the tissue.

The spring 73 further aids in allowing for a more gradual withdrawal of the cannula 34 from the tissue site in a manner that does not result in the tack 1 being disadvantageously withdrawn as well.

Two embodiments of a spring mechanism are shown in FIG. 29 by exploded views of the interior portions of the tack applicator 20. As depicted, the spring 73 and detent 73′ are located within the tapered portion of the tack applicator handle 22. In the illustrated embodiments, the spring 73 is located behind the loading tip 24, which holds the guide mechanism 74. Thus, as the tack 1 is inserted into the target tissue, pressure is applied to the tack, which, in turn, applies pressure to the carrier assembly 30. This causes the carrier assembly 30 holding the tack 1 to be pushed backwards as the spring 73 compresses.

Once the spring 73 is fully compressed, the tack 1 has reached its maximum depth and the tack applicator 20 cannot push the tack 1 any deeper into the target tissue. Also, once detent spring 73′ is fully compressed, the guide mechanism 74 cannot be pushed back into the tack applicator 20 any further. At this point, the tack applicator 20 is pulled backwards. As this occurs, the pressure of the tack 1 against the carrier assembly 30 is reduced, which, in turn, allows the springs 73, 73′ to expand back to their fully extended positions. Thus, as the tack applicator 20 holding the carrier assembly 30 is pulled backwards away from the tack 1, the springs 73, 73′ and the carrier assembly 30 with the cannula 34 in front of the spring 73, 73′ are allowed to extend somewhat towards the tack 1, thereby increasing the likelihood of proper removal of the tack 1 from within the cannula 34.

The presence of the detent spring 73′ in addition to spring 73 is preferable in that the two springs can act together to compress within the tack applicator 20 and ensure proper insertion depth of the tack 1. Alternatively, a single free-floating spring 73 may be used.

When two springs 73, 73′ are used, it is preferable to locate them behind the guide mechanism 74 and the loading tip 24 that holds the carrier assembly 30 such that as pressure is applied the carrier assembly, the guide mechanism and loading tip 24 assembly push on the springs 73, 73′ to compress them. For example, as shown in FIG. 29, the loading tip 24 may seat against the spring 73′. The guide mechanism 74 may then be attached to the loading tip 24, e.g., by sliding through a hole in the loading tip 24. Further, a pin 77 may be inserted through the loading tip 24 and the guide mechanism 74 in order to hold the pieces together. The pin 77 may further hold the carrier assembly 30 within the loading tip 24 by fitting within the notch 37 in the back of the carrier assembly.

The pin 77 may fit through either a slot 78 in the guide mechanism 74 (see FIGS. 26 and 27) or through a hole 78 in the guide mechanism 74 (see FIG. 28) in order to hold the guide mechanism and loading tip 24 together. The hole 78 shown in FIG. 28 holds the guide mechanism 74 stationary within the loading tip 24, whereas the slot 78 shown in FIGS. 26 and 27 allows the guide mechanism 74 to have an added range of motion within the loading tip 24, thereby providing additional compression of the carrier assembly 30 after the springs 73, 73′ have been completely compressed.

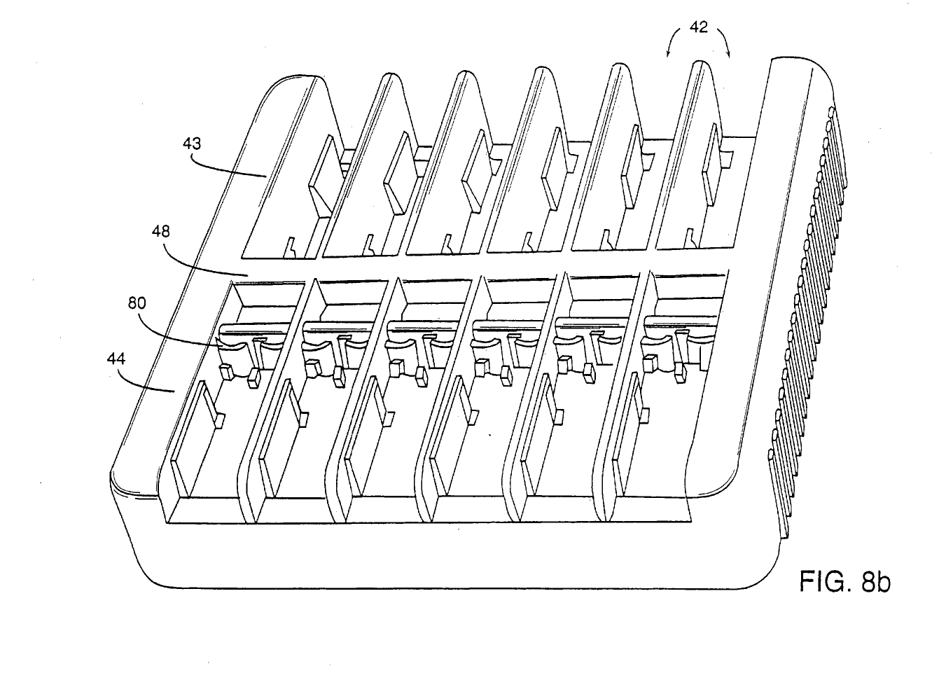

For convenience, a number of carrier assemblies 30 and tacks 1 may be mounted in a cassette 40 for easy loading and disposal of new and used carrier assemblies 30. Various views of exemplary cassettes 40 are shown in FIGS. 8, 8b and 10-13. The cassettes 40 are preferably disposable and preferably contain a plurality of channels 42 (which are preferably parallel), wherein the specific number of channels can vary from as few as one to ten or more.

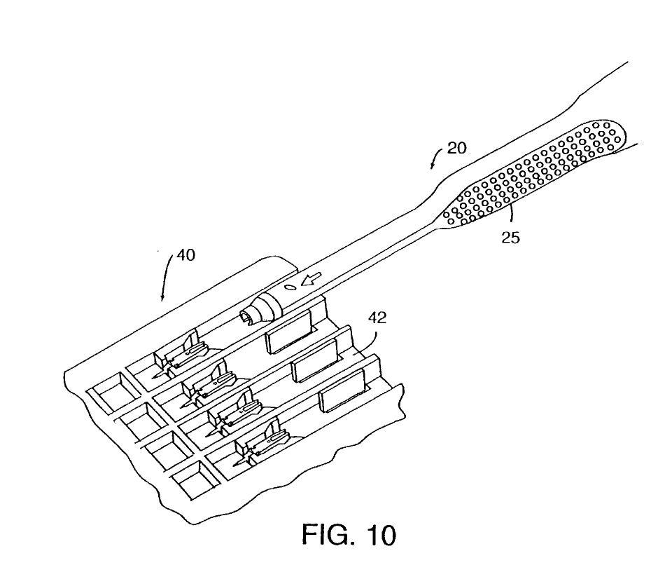

Optionally and as shown in FIG. 12, the channels 42 may be divided into two columns, wherein one column 43 is for new carrier assembly pickup and the other column 44 is for used carrier assembly disposal. Thus, after the tack 1 is released from the cannula 34 and into tissue, the carrier assembly 30 may be disposed of by loading it into the disposal column 44.

The channels 42 of the cassette 40 are preferably designed such that the tack's conical head 4 can be positioned essentially at the front end of the channel 42, and such that a space is located/defined behind the back end 37 of the carrier assembly's body portion 32. Further the tack's head 4 should be protected by leaving a sufficient opening 47 in front of the carrier assembly. Still further, and as shown in FIG. 8b, a flap 48 may be included that extends completely or partially over the gap 47 in the pickup channel 43, thereby covering the tack and providing an added safety feature, namely, as a user pushes the tack applicator 20 into the pickup channel 43, the flap 48 will prevent the user from pushing straight through the channel and puncturing himself/herself or another with the tack. 1.

Carrier assemblies 30 may be held securely within the channels 42 by the groove 37 in the carrier assembly body portion 32, as shown in FIGS. 9, 9C and 10-13. There may also be at least one indentation 39 (see FIG. 9) in the sides of the carrier assembly's body portion 32 to further lock the carrier assembly 30 within the channels 42 as shown in FIGS. 10-13. This is accomplished by providing a plurality of protrusions (not shown) in the channels 42, wherein the protrusions fit and lock into the groove 37 and the indentations 39.

The channels 42 are preferably designed such that the tack applicator 20 slides into the space behind the carrier assembly 30 and into alignment with the carrier assembly 30, thereby ensuring proper loading of the carrier assembly into the loading tip 24 of the tack applicator 20. Thus, one may load a carrier assembly into the tack applicator 20 by simply pushing the tack applicator into a channel 42 until the loading tip 24 of the applicator engages and locks onto a carrier assembly.

As shown in FIG. 11 and to further aid in proper insertion of the applicator 20 and proper pick-up of the carrier assembly 30, guides 46 may be located along the channel edges such that the tack applicator 20 slides underneath the guides 46 and into alignment with the carrier assembly 30.

Still further, corresponding notches 27 may be located on the edges of the tack applicator 20 to further aid in proper alignment of the tack applicator in the channel 42. Once the carrier assembly 30 is loaded/locked into the loading tip 24, the tack applicator 20 can then be withdrawn by lifting it out of the channel 42.

The surgical tack 1 and tack applicator 20 may be used to approximate and fix tissue (e.g., knee cartilage) and membranes (e.g., a periosteum patch) quickly and accurately during surgical procedures (e.g., Autologous Chondrocyte Implantation (ACI)). To that end, an exemplary ACI procedure is described in detail below.

Initially, predetermined area(s) of the surgical tack 1 are coated with fluid (preferably hydrophobic fluid). The tack 1 is then loaded into the cannula 34 of the carrier assembly 30 by sliding the tack into the cannula 34, back end first, such that the tail 6, transverse locking member 8, and ramp-like support 10 extend from the corresponding slot 36 and such that the tack 1 is preferably held in place via capillary forces that are induced—due to the presence of the coating fluid—by a meniscus between the tack 1 and the cannula 34, wherein the leading edge of the cannula is adjacent the proximal end 7 of conical head 4.

Next, the carrier assembly 30 is mounted into the loading tip 24 of the tack applicator 20. This is generally accomplished by mounting the back end 37 of the body portion 32 of the carrier assembly into the tack applicator 20 such that a substantial portion of the carrier assembly 30 body portion 32 is held and locked within the loading tip 24 and such that the cannula 34 and tack 1 extend out from and in front of the loading tip 24, as best seen in FIGS. 7 and 13.

In the case where a cassette 40 is used, the carrier assemblies 30—each carrying at least one tack 1—are preferably pre-mounted into the channels 42 of the pickup column 43, as shown in FIG. 8.

By virtue of this arrangement, the tack applicator 20 can be simply pushed into a channel 42 as shown in FIG. 10, until the loading tip 24 engages and locks onto a carrier assembly 30. The tack applicator 20 is then withdrawn from the channel 42 with the carrier assembly 30 in communication with the tack applicator and the tack 1 in communication with the carrier assembly. At this time, the tack 1 is ready for insertion into a target tissue site.

To that end, the tack applicator 20 is positioned such that the sharp conical head 4 of the tack 1 is at the desired location for insertion into target tissue. A user (not shown) pushes the tack applicator 20 into the target tissue with requisite force to enable the sharp head 4 of the tack 1 to penetrate the target tissue. Once the desired depth of penetration has been reached, the user gradually retracts the tack applicator 20 from the tissue. The tack's transverse locking member 8 and the proximal end 7 of the sharp conical head 4 resist backward motion out of the tissue, thereby providing enough counteracting force to overcome the capillary induced cohesive force that is maintaining the tack 1 within the cannula 34. Thus, the tack 1 will remain deployed within the target tissue upon retraction of the tack applicator 20 even if the target tissue is severely damaged and capable of providing only minimal counteracting force, and at least a portion of the target tissue will remain between the conical head 4 and the transverse locking member 8 of the tack.

Preferably, the transverse locking member 8 is offset from the longitudinal axis 3 of the head 4 and the rod 2, thus further aiding in the positioning and maintenance of the tack 1 within the desired location. Specifically, and as shown in FIG. 14, in a procedure where a temporary patch 13 (e.g., a periosteum patch) is used to repair cartilage 14 or bone by attaching the patch over the cartilage or bone defect, the location of the transverse locking member 8 above the plane of the rod 2 ensures that the tack 1 can be buried deep into the underlying cartilage while the temporary patch is kept flush or close to flush with the articular surface.

As shown in FIG. 14, the transverse locking member 8 is designed to impart a force vector on the tack 1 in a radial direction from the head 4 to the transverse locking member. This acts to pull the periosteum 13 upward and under the transverse locking member 8, from position “b” to position “a”, thus ensuring that the tack 1 can be buried deep within the cartilage or bone 14, and permitting the tack 1 to form a seal between the two objects 13, 14, wherein the seal is clean and flush or close to flush with the surface of the joined objects.

If a cassette 40 is used, then after insertion of the tack 1 into the site, the used carrier assembly 30 may be discarded in a used carrier assembly disposal channel 44 of the cassette by pushing the tack applicator 20—loading tip 24 end first—into the disposal channel. The disposal channel 44 includes means for latching onto and gripping the used carrier assembly 30 and for disengaging the carrier assembly from the loading tip 24 as the tack applicator 20 is backed out of the channel 44.

Once a used carrier assembly 30 is disengaged/detached from the tack applicator 20, the tack applicator can be reloaded, e.g., in furtherance of loading an additional carrier assembly into which a tack 1 is loaded for subsequent placement—using the tack applicator—at the same target tissue site or a different target tissue site.

Tacks 1 and tack applicators 20 as described above may be used in open procedures, and also in less invasive arthroscopic procedures. The overall dimensions and shape of the tack applicator 20 and carrier assembly 30 will vary depending on the particular type of procedure.

Arthroscopic procedures are known, and generally involve the use of trocars, or other hollow delivery mechanisms, through which the various tools required during the procedure are inserted into the site. These trocars and other hollow delivery mechanisms are typically narrow elongate tubes that may be straight or curved, relatively flexible or rigid. The hollow delivery mechanisms are inserted through a small incision made near the site.

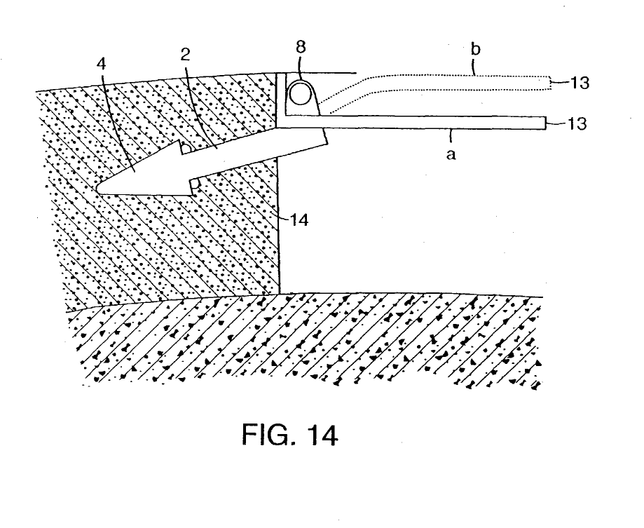

For example, as shown in FIGS. 16-20, surgical tacks 1 and applicators 20 of the present invention can be utilized in accordance with one method of arthroscopically repairing cartilage in the knee. Such a method generally entails accessing and preparing the surgical site, followed by fixing a natural or synthetic membrane patch, such as a periosteal patch, over the cartilage defect at the site.

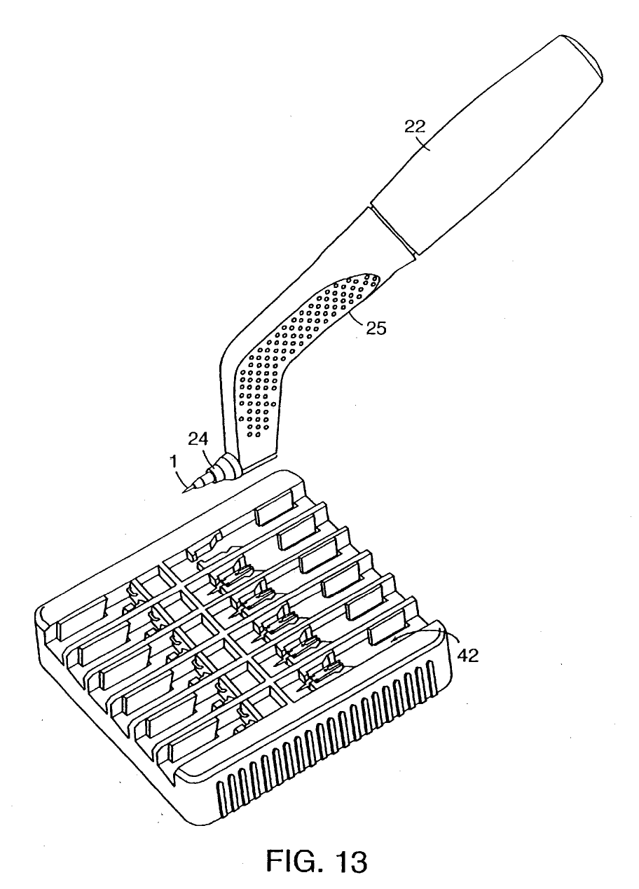

In arthroscopic procedures involving the right knee, the defect is accessed through any of the portal sights of the knee, shown in FIG. 15, which include the superolateral portal 50, lateral midpatellar portal 51, anterolateral portal 52, lateral auxiliary portal 53, lateral parapatellar tendon portal 54, central transpatellar tendon portal 55, superomedial portal 56, medial midpatellar portal 57, anteromedial portal 58, medial auxiliary portal 59, and the medial parapatellar tendon portal 60. Similar portals and procedures may be used on the left knee.

A number of trocars or other hollow delivery mechanisms 62 may then be positioned for access to different areas of the knee. For example, FIG. 16 illustrates an exemplary portal usage combination for repairing a medial defect, wherein three portal sites are utilized in combination: (1) the central transpatellar tendon portal 55, which may be used for insertion of a first hollow delivery mechanism 62, (2) the superomedial portal 56, which may be used for insertion of a second hollow delivery mechanism 62, and (3) the anteromedial portal 58 or the medial auxiliary portal 59, which may be used for inserting a third hollow delivery mechanism 62.

After accessing the site, the synovium is cleared away and the defect is then evaluated by conventional methods, such as by probing it with a nerve hook and by gouging it to determine the cartilage thickness. Next, the defect is circumscribed by conventional techniques. The cartilage is then debrided (using any of the conventional tools known in the art) to clean up the edges of the cartilage and to form clean, perpendicular sidewalls. The defect is then measured for patch sizing.

A template is next created using one of the known alternatives available, such as simple measurement, pressure sensitive films and 3-D digitizers. Blood from the site is cleared out, and additional bleeding is stopped by sealing any so-called “bleeders.” All of the above steps are carried out arthroscopically by inserting the appropriate tools through the hollow delivery mechanisms 62.

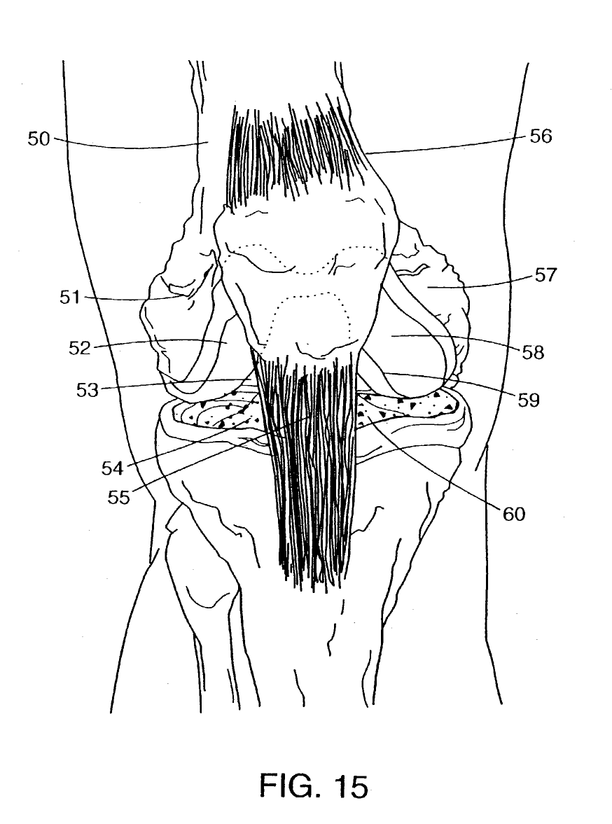

Once bleeding has been stopped, the patch may be transported by any conventional method, such as pulling the patch through one of the delivery mechanisms 62, or via an alternate technique, such as transporting the patch in a rolled mesh carrier. As illustrated in FIG. 17, the patch 64 is first placed in the center of a mesh carrier backing 65 with the cambium side up.

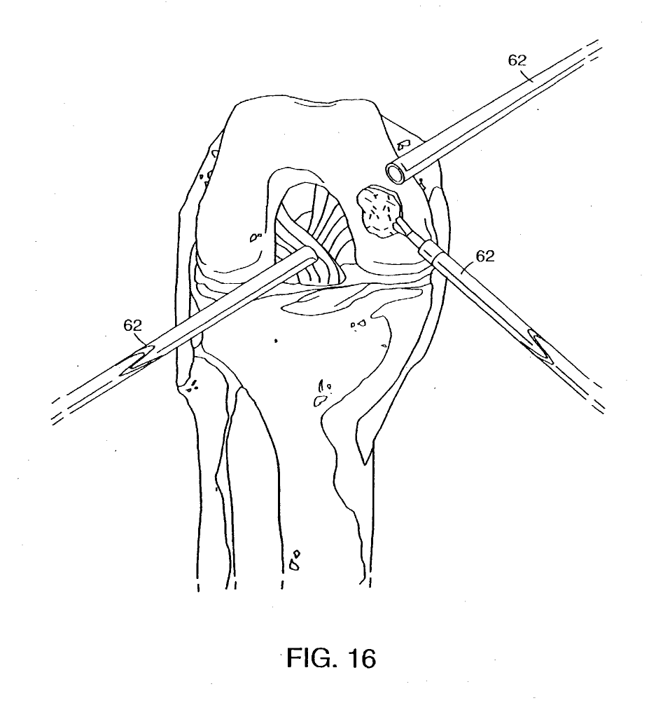

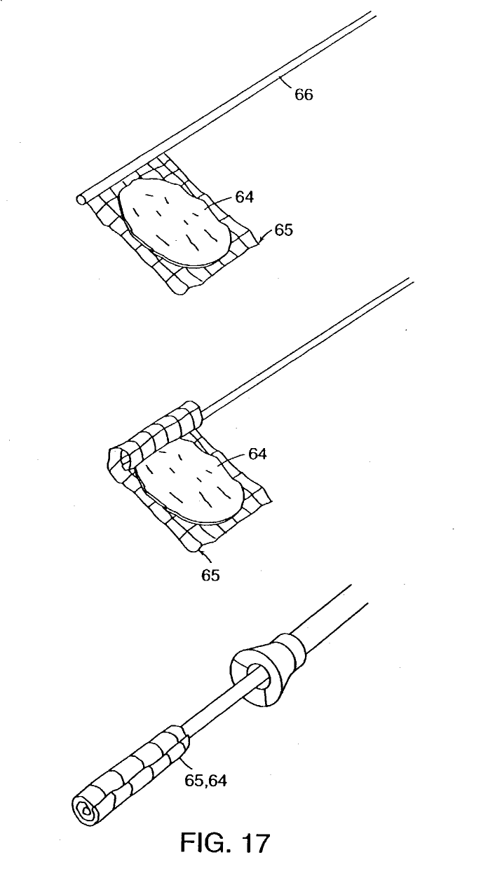

The carrier and patch are then rolled to form tight wraps by using, for example, a mandrel 66. The rolled mesh 65 and patch 64 may then be inserted into the delivery tube 62 and transferred to the site, where the mesh and patch are unrolled and the patch is positioned over the defect, as shown in FIG. 18. The patch may then be fixed to the site using the tacks 1 and applicators 20 of the present invention, as shown in FIG. 19, and as discussed above.

If Autologous Chondrocyte Implantation (ACI) is utilized, then prior to fixing the patch to the site, a biopsy tool is inserted through one of the hollow delivery mechanisms 62 to remove a biopsy of healthy cartilage tissue. The healthy tissue sample is then cultured externally. After the site is prepared as such, the patch is fixed to cover the lesion by the above-described procedure. The cultured chondrocytes are then delivered into the lesion beneath the patch. Within the lesion, the cultivated cells produce a matrix, which integrates with the surrounding cartilage. Over time, the cells continue to mature and fill in the lesion with healthy cartilage.

Although the overall design of the tacks and the tack applicators are generally the same for both the open and arthroscopic procedures, due to the nature of arthroscopic procedures, a narrower and longer applicator assembly is generally required for insertion in accordance with the hollow delivery mechanism. For example, an extended carrier assembly 30 may be used. This mechanism may consist of a similar slotted 36 cannula 34 that is straight or curved. Also, the cannula 34 may be mounted in a longer and more narrow carrier assembly 30 than the carrier assemblies used in open procedures.

As shown in FIG. 21, the carrier assembly 30 may further have a cross section that is essentially rectangular, oval shaped, round with a notch or raised area, or any other shape that assists in maintaining proper orientation of the tack 1 within the carrier assembly.

In some applications (for example, FIG. 21), the carrier assembly 30 may further be made of flexible or kerfed material to facilitate its delivery through a tube, a curved trocar or another hollow delivery mechanism 62 with a similarly shaped cross section. This delivery mechanism 62 may be curved at its distal end to allow for improved access to some joint areas.

The tack applicator 20 and carrier assembly 30 may also allow for rotation of the delivery mechanism 62 in order to further improve joint access while still tracking the orientation of the tack 1. This type of assembly would allow replacement of carrier assemblies 30 with tacks 1 without losing location (triangulation) within the joint. It is also possible to utilize a carrier assembly 30 without a trocar 62, based on medical discretion.

Once the target tissue site is prepared and the patch is ready for fixation, the tack 1 is loaded into the cannula 34 of the carrier assembly 30 and the carrier assembly 30 is mounted within the loading tip 24 of the tack applicator 20 as described above.

The tack applicator 20 is inserted through a portal, such as the anteromedial portal 58 or the medial auxiliary portal 59, and positioned such that the sharp conical head 4 of the tack 1 is at the desired location for insertion into the patch 64. At the same time, and as shown in FIG. 19, the patch 64 may be held by a grasping device 67.

Preferably, a plurality of tacks 1 are inserted along the periphery of the patch, close to the edges of the patch 64. A camera or viewing device (not shown) may be inserted through delivery mechanism 62 to provide the user with an enhanced view in order to assist in achieving proper placement of the tack(s) 1.

In accordance with the present invention, tacks 1 are designed to penetrate the patch 64. Due to the small size of the tacks, there is a beneficially diminished possibility of tearing through the edges of the patch 64. The conical head 4 of the tack 1 penetrates through the patch 64 and into the underlying cartilage. The tack 1 is preferably inserted such that the conical head 4 of the tack does not penetrate through the cartilage, but rather, remains buried within the cartilage, as shown in FIG. 14. Once a desired tack 1 depth had been reached, the user gradually retracts the tack applicator 20 from the portal.

The tack's transverse locking member 8 and the proximal end 7 of the sharp conical head 4 individually and collectively cause resistive forces to be exerted upon the tack 1, wherein these forces—even if the cartilage is damaged—are more than enough to overcome the clearance fit and capillary forces that exist between the tack 1 and the cannula 34 due to the presence of a meniscus (as described herein), thus ensuring that the tack 1 is disengaged from the cannula and remains within the cartilage. The conical head 4 and the transverse locking member 8 also hold the tack 1 securely within the cartilage and ensure that the patch 64 is flush or close to flush against the cartilage.

The preferably rounded edges of the transverse locking member 8 provide a gentle engagement with the delicate patch in order to resist tearing of the patch 64. Because the transverse locking member 8 is offset from the longitudinal axis 3 of the head 4 and the rod 2, the positioning of the tack 1 in its desired location is facilitated, as is the maintenance of the tack in/at that position. And the location of the transverse locking member 8 above the plane of the shaft ensures that the tack 1 can be inserted deep into the underlying cartilage and, in addition, ensures that the patch 64 is kept flush or close to flush with the articular surface of the cartilage, as shown in FIG. 14.

Once the tack 1 has been inserted as such, the used carrier assembly 30 may be disposed of or discarded, e.g., in a used carrier assembly disposal channel 44 located within a cassette 42. Additional tacks 1 can then be inserted into/about the defect site until the patch is satisfactorily fixed over the defect site, with each subsequent insertion process generally resembling the process described above.

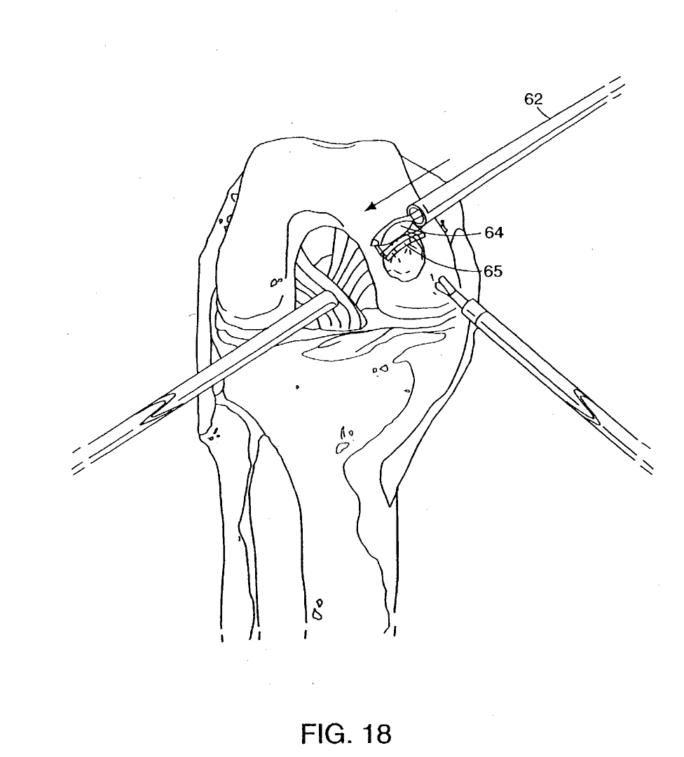

After the tacks 1 are inserted about the patch 64 periphery, excess edges of the patch 68 may be trimmed flush or close to flush to the edge of the defect using, for example, angle scissors 69 inserted through tube 62, as shown in FIG. 20.

Although the present invention has been described herein with reference to specific details of preferred embodiments thereof, it is not intended that such details should be regarded as limiting the scope of the invention, except as and to the extent that they are included in the accompanying claims. Moreover, any documents mentioned herein are incorporated by reference in their entirety.

1. A surgical fastening assembly comprising:
an applicator including a loading tip;
a carrier assembly removably mounted within the loading tip;
a cannula having an inner diameter and an outer diameter, the cannula being disposed within the carrier assembly; and
a surgical fastener disposed at least partially within the cannula such that a predetermined distance is defined between the surgical fastener and the inner diameter of the cannula, wherein at least a portion of the surgical fastener is in communication with a fluid, and wherein the presence of the fluid defines a meniscus that induces a capillary force between the surgical fastener and the inner diameter of the cannula.

2. The surgical fastening assembly of claim 1, wherein the fluid is a biocompatible hydrophobic fluid.

3. The surgical fastening assembly of claim 1, wherein the predetermined distance is selected to enable a clearance fit to be formed between the surgical fastener and the inner diameter of the cannula.

4. The surgical fastening assembly of claim 3, wherein the predetermined distance is between about 0.0025 inch and 0.006 inch.

5. The surgical fastening assembly of claim 4, wherein the predetermined distance is about 0.003 inch.

6. The surgical fastening assembly of claim 1, wherein the surgical fastener includes at least one positive surface feature, and wherein the fluid is in communication with at least one of the at least one positive surface feature.

7. The surgical fastening assembly of claim 6, wherein the presence of the at least one positive surface feature reduces the predetermined distance by approximately 66% at the at least one positive surface feature.

8. The surgical fastening assembly of claim 1, wherein the surgical fastener is formed from a bioabsorbable material.

9. A method of introducing a surgical fastener into an implantation site, comprising the steps of:
providing a surgical fastener;
providing a cannula having an inner diameter and an outer diameter;
placing fluid into communication with at least a portion of the surgical fastener;
at least partially introducing the surgical fastener into the cannula such that the portion of the surgical fastener that is in communication with the fluid is disposed within the cannula and such that a predetermined distance is defined between the surgical fastener and the inner diameter of the cannula;
allowing the presence of the fluid to define a meniscus that induces a capillary force between the surgical fastener and the inner diameter of the cannula; and
causing the surgical fastener to be deployed from within the cannula and into an implantation site.

10. The method of claim 9, wherein the fluid is a biocompatible hydrophobic fluid.

11. The method of claim 9, wherein the predetermined distance is selected to enable a clearance fit to be formed between the surgical fastener and the inner diameter of the cannula.

12. The method of claim 11, wherein the predetermined distance is between about 0.0025 inch and 0.006 inch.

13. The method of claim 12, wherein the predetermined distance is about 0.003 inch.

14. The surgical fastening assembly of claim 9, wherein the surgical fastener includes at least one positive surface feature, and wherein the fluid is in communication with at least one of the at least one positive surface feature.

15. The surgical fastening assembly of claim 14, wherein the presence of the at least one positive surface feature reduces the predetermined distance by approximately 66% at the at least one positive surface feature.

16. The method of claim 9, wherein the step of placing fluid into communication with at least a portion of the surgical fastener is at least partially accomplished through the use of an applicator.

17. The method of claim 16, wherein the applicator is selected from the group consisting of a brush-type applicator and a spray-type applicator.

18. The method of claim 9, wherein the step of placing fluid into communication with at least a portion of the surgical fastener is at least partially accomplished through dipping the portion of the surgical fastener into a containment element that contains the fluid.

19. The surgical fastening assembly of claim 9, wherein the surgical fastener is formed from a bioabsorbable material.
